(19) United States
(12) Patent Application Publication
(10) Pub. No.: US 2026/0355462 A1
(43) Pub. Date: Nov. 12, 2026

(54) TWIN LEAD SCREW FOR CATHETER ROBOTIC SYSTEM

(71) Applicant: Covidien LP, Mansfield, MA (US)

(72) Inventor: Robert H. White, Roslindale, MA (US)

(73) Assignee: Covidien LP, Mansfield, MA (US)

(21) Appl. No.: 19/673,712

(22) Filed: May 11, 2026

Related U.S. Application Data
(60) Provisional application No. 63/803,275, filed on May 9, 2025.

(51) Int. Cl. A61M 25/01; A61B 34/30

(52) U.S. Cl. CPC A61M 25/0113; A61B 2034/301; A61M 2205/103

(57) ABSTRACT

A catheter articulation system including a holder, a motor configured to be secured to the holder, a lead screw connected to the motor and including a first threaded portion having a first major diameter and a second threaded portion having a second smaller major diameter, a first follower having internal threads configured to advance or retract along the first threaded portion, and a second follower having internal threads configured to advance or retract along the second threaded portion.

### FIELD

This disclosure relates to surgical systems for catheter navigation. More particularly, the present disclosure relates to a twin lead screw system and methods for articulating motor driven and robotic cardiac catheters.

### BACKGROUND

More and more surgeons are utilizing surgical robots to drive endoscopes, endoscopic instruments, and other catheter-based devices providing access to organs through natural orifices or small incisions. Traditionally, catheter systems have been manually navigated through, for example, the femoral artery or vein or another vein or artery to arrive at a desired location in or near the heart. Often these manually navigated systems require the use of guide wires to assist in the navigation. Further, where the manually navigated systems are articulable, effective articulation of the catheter in more than one plane to achieve a desired orientation and shape is challenging. While some robotically catheter systems have been developed, these are often dedicated to just a single type and size of catheter and often offer significant challenges in the set-up prior to the procedure. Still further, protection of the drive systems and maintaining sterility of the operating environment present additional challenges for robotic and powered catheter drive systems. This disclosure is directed at addressing the shortcomings of prior systems and overcoming these challenges.

### SUMMARY

One aspect of the disclosure is directed to a catheter articulation system including a holder; a motor configured to be secured to the holder, a lead screw connected to the motor and including a first threaded portion having a first major diameter and a second threaded portion having a second smaller major diameter, a first follower having internal threads configured to advance or retract along the first threaded portion, and a second follower having internal threads configured to advance or retract along the second threaded portion.

Implementations of this aspect may include one or more of the following features. The catheter articulation system where the first follower is advanceable over the second threaded portion without interacting with the threads of the second threaded portion. Rotation of the lead screw advances the first follower along the first threaded portion and the second follower along the second threaded portion a same longitudinal distance. Rotation of the lead screw in a first direction advances the first follower along the first threaded portion in a first direction and advances the second follower along the second threaded in a second direction opposite the first direction. Rotation of the lead screw in a second direction advances the first follower along the first threaded portion in a second direction and advances the second follower along the second threaded in a first direction opposite the second direction. The second threaded portion has a different number of starts than the first threaded portion. The second threaded portion has a different pitch than the first threaded portion. The first follower and the second follower slide over the rail as they are advanced or retracted along the first threaded portion and the second threaded portion. The catheter articulation system may include a first bearing and a second bearing and configured to receive and support the lead screw. The first follower and the second follower are configured to engage tabs of a catheter to move the tabs towards or away from one another to articulate the catheter in a first plane.

A further aspect of the disclosure is directed to a navigational catheter system including a catheter drive system configured to receive a catheter, the catheter drive system including: a holder, a motor configured to be secured to the holder, a lead screw connected to the motor and including a first threaded portion having a first major diameter and a second threaded portion having a second smaller major diameter, a first follower having internal threads configured to advance or retract along the first threaded portion, a second follower having internal threads configured to advance or retract along the second threaded portion. The system also includes a mount connected to the catheter drive system and configured to enable adjustment of an orientation of the catheter drive system; and a base, removably connectable to the mount, the base enabling adjustment of a position of the catheter drive system. Other embodiments of this aspect include corresponding computer systems, apparatus, and computer programs recorded on one or more computer storage devices, each configured to perform the actions of the methods.

Implementations may include one or more of the following features. The navigational catheter system where the first follower is advanceable over the second threaded portion without interacting with the threads of the second threaded portion. Rotation of the lead screw advances the first follower along the first threaded portion and the second follower along the second threaded portion a same longitudinal distance. Rotation of the lead screw in a first direction advances the first follower along the first threaded portion in a first direction and advances the second follower along the second threaded in a second direction opposite the first direction to articulate the catheter in first direction in a first plane. Rotation of the lead screw in a second direction advances the first follower along the first threaded portion in a second direction and advances the second follower along the second threaded in a first direction opposite the second direction to articulate the catheter in a second direction in the first plane. The first follower and the second follower are configured to engage tabs of the catheter to move the tabs towards or away from one another to articulate the catheter in the first direction or the second direction in the first plane. The second threaded portion has a different number of starts than the first threaded portion. The second threaded portion has a different pitch than the first threaded portion. The first follower and the second follower slide over the rail as they are advanced or retracted along the first threaded portion and the second threaded portion. The navigational catheter system may include a single bearing configured to receive and support the lead screw.

### BRIEF DESCRIPTION OF THE DRAWINGS

FIG. 1 depicts a top view of a catheter drive system and catheter in accordance with the disclosure;

FIG. 2 depicts a side view of the catheter drive system and catheter of FIG. 1;

FIG. 3 is a perspective view of a known lead screw system; and

FIG. 4 is a perspective view of a twin lead screw system in accordance with the disclosure.

### DETAILED DESCRIPTION

This disclosure is directed to systems and methods for catheter navigation, and particularly the motorizing or robotizing a catheter-based system for cardiac navigation. The systems and methods of this disclosure reduce the number of components required for catheter articulation and improves efficiency in the assembly of the articulation system.

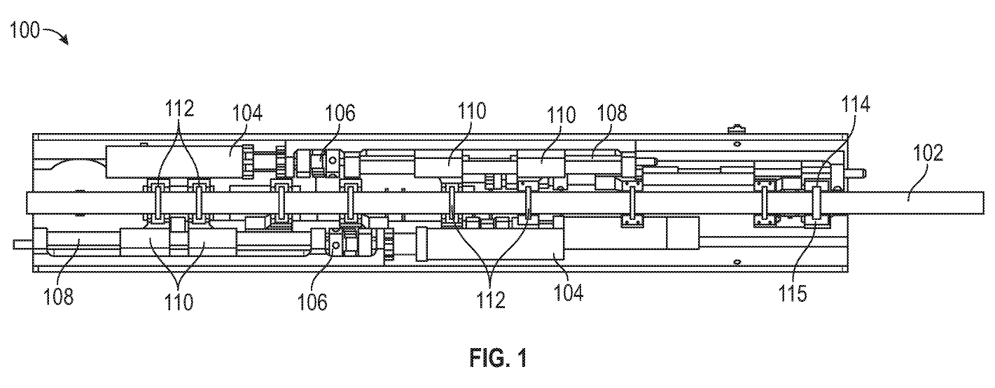

FIG. 1 is a top view of a catheter drive system 100 in accordance with the disclosure. The catheter drive system 100 is configured to have a catheter 102 dropped into the catheter drive system 100. The catheter drive system 100 includes a plurality of motors 104 (e.g., stepper motors). Each motor 104 is connected via a coupling 106 to transfer rotational motion from the motor 104 to a lead screw 108 with two oppositely threaded sections (shown graphically here without their threads). A pair of followers 110, which include matching and mating threads to the lead screw 108, are mounted on the lead screw 108. The lead screw 108 has two separate sections of threads, which are oppositely threaded, thus by rotation of the shaft extending from the motor 104 in a first direction the followers 110 are drawn towards one another along the lead screw 108 and rotation of the shaft in a second direction causes the followers 110 to move away from one another. Each follower 110 mates to a tab 112 of the catheter 102. Though shown and described herein with four motors 104, the disclosure is not so limited and one, two, five, six, and other configurations of motors 104 may be employed without departing from the scope of the disclosure.

Each tab 112 may connect to a pull wire or tendon (not shown) within the catheter 102. In accordance with one aspect of the disclosure, to effectuate articulation of the catheter 102 in one plane two pull-wires or tendons may be employed, with a first pull wire or tendon being retracted and a second opposing pull-wire or tendon being extended. Use of two pull wires or tendons promotes controlled articulation movements of the catheter 102. The tabs 112 connected to oppositely threaded portions of the lead screw 108 via the followers 110 move in tandem and in opposite directions with limited slop or backlash (e.g., limited a clearance or lost motion caused by gaps between the threads of the lead screw 108 and threads of the followers 110).

As will be appreciated, articulation in a single plane can be achieved with a single pull-wire or tendon being acted on by a single tab 112 and follower 110. To return to an unarticulated position, the pull wire may be driven by the tab 112 and follower 110 in the opposite direction as to articulate the catheter 102. In some instances, the material of the catheter 102 may assist in returning the catheter to an orientation that is unbiased by the pull wire. Though not described in great detail herein, such configurations may be employed without departing from the scope of the disclosure.

In accordance with one aspect of the disclosure, the catheter drive system 100 includes four motors 104, four lead screws 108, and four pairs of followers 110. The arrangement is configured to mate with a catheter 102 including an inner catheter and an outer catheter, each of the inner catheter and outer catheter include two pairs of tabs 112, such that each of the inner catheter and outer catheter can be articulated in two planes (e.g., X and Y) and can achieve nearly any angle of articulation between these planes by independent and coordinated actuation of the tabs 112 for articulation on the X plane and the tabs 112 for articulation in the Y plane.

The stiffness of the catheter 102, and its resistance to movement (e.g., from forces applied to the catheter 102 by the body) may be adjusted by the independent actuation of the motors 104. Still further, the lead screws 108 may be configured to prevent back-driving of the catheter and to have minimal backlash or slop in their connection to the followers 110 by selection of the thread patterns of the lead screws 108 and followers 110.

A thrust surface 114 (or thrust bearing) formed on the catheter 102 serves multiple purposes. First, the thrust surface 114 is received in a bearing recess 115 of the catheter drive system 100, and thus prevents axial movement of the catheter 102 relative to the catheter drive system 100, while permitting the tabs 112 of the catheter 102 and their attendant pull wires or tendons to freely move within the catheter drive system 100. In addition, an optional fifth motor (not shown) operably connects to the thrust surface 114 (e.g., via a gear, also not shown) and enables rotation of the catheter 102 within the catheter drive system 100. The tabs 112 and thrust surface 114 encircle the catheter 102, thus despite rotation of the catheter, the tabs 112 remain operably connected to the followers 110 and the thrust surface 114 remains operably connected to its motor.

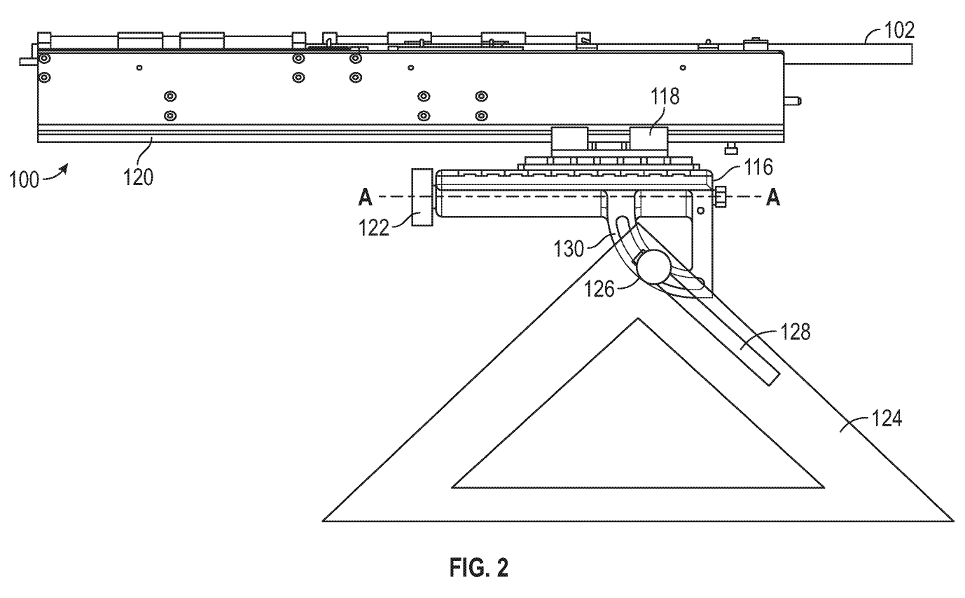

Further aspects of the catheter dive system 100 are depicted in FIG. 2. As shown, the catheter drive system 100 includes a mount 116. The mount 116 includes linear drive 118 which operably connects to a rail 120. The combination of the linear drive 118 and rail 120 enable the advancement of the catheter 102 and the catheter drive system 100 along the axis defined by the rail 120 relative to the mount 116. A knob 122 formed on the mount 116 can be loosened to allow the catheter drive system 100 to be rotated about an axis A relative to the mount 116, retightening of the knob secures the catheter drive system 100 relative to the mount 116 at the desired angle of rotation about axis A.

A base 124 is placed on a procedure table and is operably and releasably connected to the catheter drive system 100 via the mount 116. While the base 124 is depicted as a generally triangular structure, alternative shapes and designs for the base 124 may be employed. A knob 126 is part of a securing mechanism connecting the base 124 and the mount 116. Loosening of the knob 126 allows the mount 116 and the attached catheter drive system 100 to be moved along slot 128 formed in the base 124 to adjust the both the axial position, and the height, of the catheter drive system 100 relative to the procedure table on which the base 124 is placed. Loosening of the knob 126 also allows for the mount 116 and therewith the catheter drive system 100 to be rotated relative to the base 124 along the arc defined by slot 130 formed in the mount 116. Through the use of the knob 122, knob 126, and slots 128 and 130, the height, pitch, and the roll of the catheter drive system 100 (and therewith the catheter 102) relative to the procedure table and the patient can be adjusted.

The base 124 (e.g., via the knob 126) allows for the quick and easy connection and disconnection of the catheter drive system 100. By disconnecting the catheter drive system 100, the catheter 102 may be manually inserted into the patient and navigated to a desired location. After manual navigation, the catheter drive system 100 can be connected to the base 124, and the catheter 102 can be inserted into the catheter drive system 100. Once the catheter 102 is inserted into the catheter drive system 100, the catheter can be robotically navigated through subsequent portions of the patient’s anatomy (e.g., to positions and orientations within chambers of the heart).

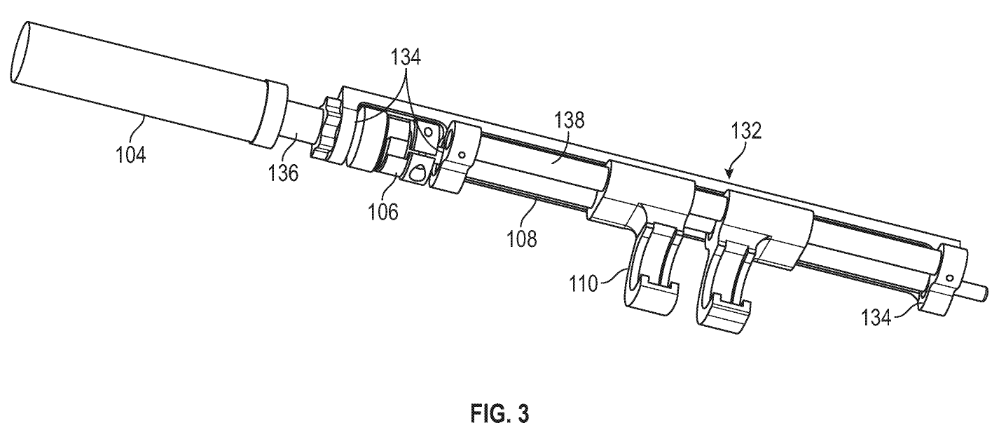

As depicted in FIG. 1, the catheter drive system includes multiple motors 104, connected via a coupling 106 to a lead screw 108 to drive the followers 110 to articulate the catheter 102. A more specific example of this arrangement for articulating the catheter 102 is depicted in FIG. 3. In FIG. 3 the motor 104 mounts to a holder 132, the coupling 106 connects the motor 104 to the lead screw 108. Bearings 134 within the holder 132 support the lead screw 108 on both ends and also support the output shaft 136 of the motor 104. The followers 110 are supported on and slide along a rail 138 to maintain their alignment of the followers 110 with respect to the holder 132 and the catheter 102. Though only shown graphically, as noted above, the lead screw 108 includes two sets of threads, each acting on one of the followers 110. The two sets of threads are opposite one another such that the two followers 110, following the threads as the driven by the motor 104, always move in opposite directions from one another. Thus, clockwise rotation of the lead screw 108 causes the followers 110 to move towards one another and counterclockwise rotation of the lead screw 108 causes the followers to move away from one another. In the configuration of FIG. 3, the threads of the two sets of threads on the lead screw 108 are of the same diameter and have the same thread pitch, just formed for opposing thread angles drive (e.g., right-hand treading vs left-hand threading).

While the configuration of FIG. 3 is effective, it presents a variety of challenges including requiring additional components (e.g., coupling 106, and bearings 134). Another challenge is that the overall length of the assembly is increased, making the catheter drive system 100 larger and more challenging to maneuver into place with respect to the patient. Still further, during the assembly it is impossible to pass the follower that is to be closest to the motor 104 over the oppositely threaded portion of the lead screw 108.

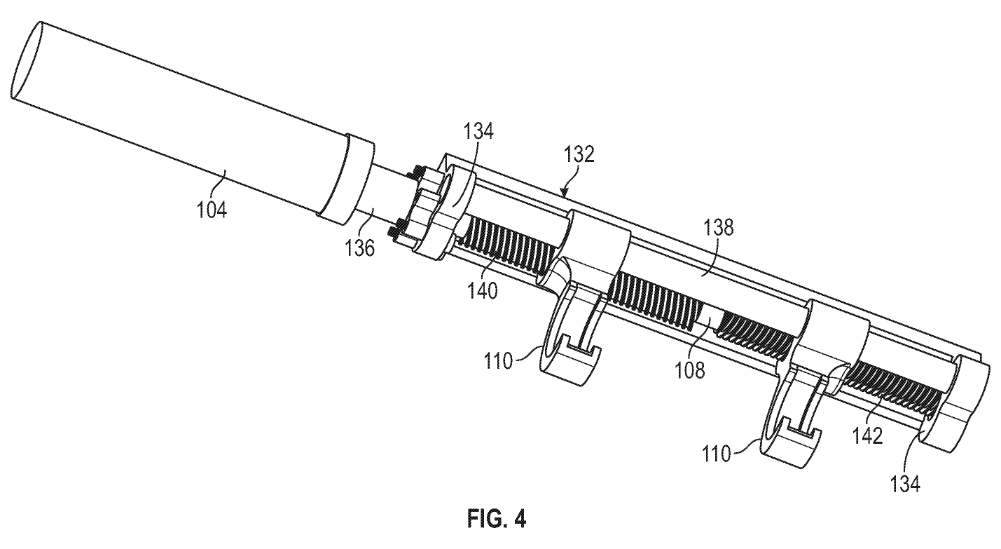

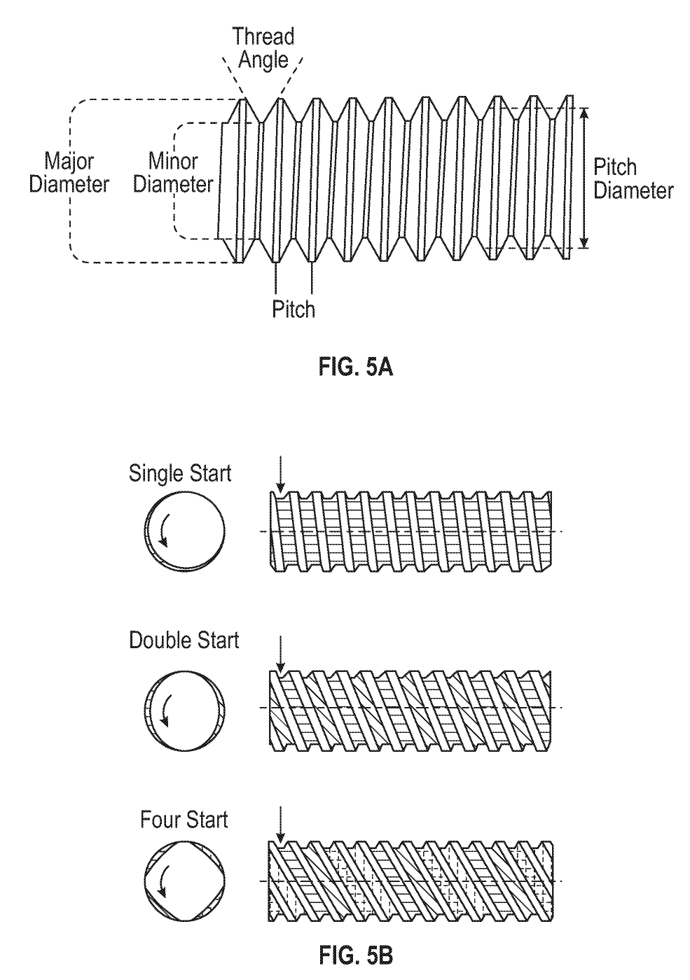

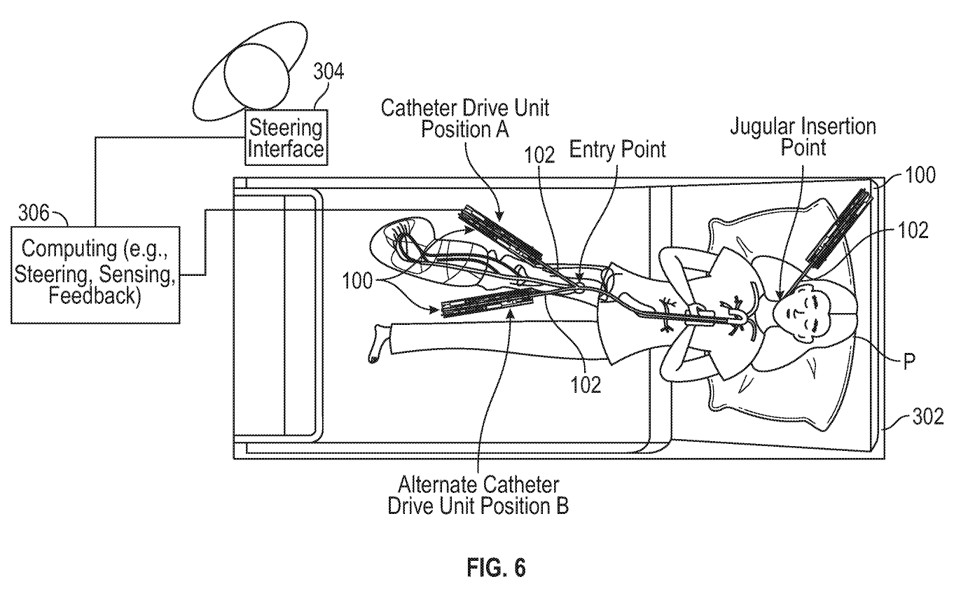

FIG. 4 depicts a solution to the challenges of the arrangement of FIG. 3. As is immediately apparent, the assembly in FIG. 4 does not include a coupling 106, instead, the shaft 136 extends through a bearing 134 on the holder 132, and itself is threaded forming the lead screw 108. The lead screw 108 has two separate sections, a first section 140 includes threads having a first diameter and direction (e.g., right hand thread), and a second section 142 having a second smaller diameter and direction (e.g., left hand thread). A first follower 110 has internal threads configured to mate with the threads of the first section 140 and a second follower has internal threads configured to mate with the threads of the second section 142. Thus, during assembly the first follower 110 can be passed over the second section 142 of the lead screw 108 without interacting with the threads on the second section 142 and placed in contact with the threads on the first section 140. Rotation of the lead screw 108 in a first direction advances the follower over the threads of the first section 140 until the follower abuts the holder 132. The second follower 110 can then be placed at an end of the second section 142 of the threads. Rotation of the lead screw 108 in second direction advances the second follows over the second section 142 due to the mating interface with the internal threads of the second follower 110. Rotation of the lead screw 108 in the second direction drives the two followers together near a center point of the lead screw 108.

Less obvious than the omission of the coupling 106, the assembly of FIG. 4 also enables the elimination of one of the bearings 134. As a result of the elimination of the coupling and the portion of the holder 132 that would house the third bearing 134, the overall length of the assembly of FIG. 4 is about 20-25% shorter than that of FIG. 3. Further, by elimination of multiple components, the overall component cost of the assembly is reduced. Still further, the procedure for assembling the components is shortened.

Though not depicted in FIG. 4, one of ordinary skill in the art will recognize that the shaft 136 may terminate in a gear head that couples to a complementary gear secured to lead screw 108. Based on a gear ratio between the gear head on the shaft 136 and the gear on the lead screw 108, the lead screw 108 can be driven at a higher or lower number of rotations per minute (RPM) than the motor 104. Further, the gears can in combination with the threads on the lead screw prevent back driving of the motor 104.

As will be appreciated, due to the difference in diameter for the first section 140 of the threads and the second section 142 of the threads of the lead screw 108 will require different threads to be machined on the first section 140 and second section 142. The threads of the second section 142 will necessarily have a major diameter so that the first follower can pass over the second section 142 without interacting with the threads. With reference to FIG. 5A the pitch is the distance between the two threads, while the major diameter is the diameter at the apex of the threads, and the minor diameter is the diameter at the trough of the threads. FIG. 5B depicts threads having different start numbers. Starts refers to the number of separate thread spirals here shown as one, two or four starts. The lead (distance traveled per rotation) along a threaded section is the pitch, the distance between two threads multiplied by the number of starts. Thus, a follower 110 on a first threaded section 140 with a single start will only move half as far as a follower 110 on a second threaded section 142 having the same pitch but two starts. With these differences in geometry considered, the threads on the first section 140 and the second section 142 can be machined such that the lead (the distance travelled per revolution) of the first section 140 is the same as the second section 142, despite the differences in major diameter of the two threaded sections 140, 142. This can be achieved through machining different major and minor diameters, pitches, or different numbers of starts in the two threaded sections 140, 142.

FIG. 6 depicts a patient P on a procedure table 302. Three different position options for placing the catheter drive system 100, coupled to the base 124, are depicted. A first position is located outside of the patient’s right leg. A second position is between the patient’s legs. Both of the first and second positions place the catheter 102 such that the catheter 102 can be inserted into the preferred entry point for accessing the heart (e.g., the femoral vein or saphenous vein). A third position is shown near the patient’s head, where the jugular vein in the patient’s neck can be accessed.

Regardless of where the catheter drive system 100 is placed relative to the patient, advancement, and control of the catheter 102 is managed via a steering interface 304. As an example, the steering interface may be a gaming controller or the like where inputs from the clinician are received by the gaming controller and converted to signals for ultimately controlling the motors 104 and linear drive 118. The signals generated by the steering interface 304 are received by a computing system 306.

The computing system 306 includes a memory and a processor. The processor is configured to execute one or more applications stored in the memory. The computing system 306 may include a display to depict various aspects of the navigation in one or more user interfaces. For example, the user interface may depict a representation of the catheter 102 as it navigates the heart. This representation of the catheter 102 may be depicted within a three-dimensional model of a portion of the patient’s anatomy (e.g., the heart).

Though not shown, the catheter 102 may include one or more sensors to detect a position and orientation of a distal portion of the catheter (e.g., electromagnetic sensors, Fiber-Bragg sensors, ultrasound sensors). In addition, data from the drive motors (e.g., motor rotations, current, voltage, tab 112 relative positions) can be utilized to both sense a shape and orientation of a distal portion of the catheter 102. This data can be used to provide additional feedback regarding resistance to movement experienced by the catheter 102, effective articulation (e.g., by comparison with articulation measured via imaging), and other informative aspects useful for the surgeon during the procedure. In some instances, this feedback can be processed by the computing system 306, and signals can be sent to the steering interface 304 to generate one or more forms of haptic feedback or may present visual and audible feedback to clinician via the computing system 306.

### Examples

This disclosure is further described in connection with the following examples, in which:

Example 1 – is a catheter articulation system including a holder, a motor configured to be secured to the holder, a lead screw connected to the motor and including a first threaded portion having a first major diameter and a second threaded portion having a second smaller major diameter, a first follower having internal threads configured to advance or retract along the first threaded portion, and a second follower having internal threads configured to advance or retract along the second threaded portion.
Example 2 – The catheter articulation system of example 1, wherein the first follower is advanceable over the second threaded portion without interacting with the threads of the second threaded portion.
Example 3 – The catheter articulation system of any of the preceding examples, wherein rotation of the lead screw advances the first follower along the first threaded portion and the second follower along the second threaded portion a same longitudinal distance.
Example 4 – The catheter articulation system of any of the preceding examples, wherein rotation of the lead screw in a first direction advances the first follower along the first threaded portion in a first direction and advances the second follower along the second threaded in a second direction opposite the first direction.
Example 5 – The catheter articulation system of any of the preceding examples, wherein rotation of the lead screw in a second direction advances the first follower along the first threaded portion in a second direction and advances the second follower along the second threaded in a first direction opposite the second direction.
Example 6 – The catheter articulation system of any of the preceding examples, wherein the second threaded portion has a different number of starts than the first threaded portion.
Example 7 – The catheter articulation system of any of the preceding examples, wherein the second threaded portion has a different pitch than the first threaded portion.
Example 8 – The catheter articulation system of any of the preceding examples, further comprising a rail secured in the holder, wherein the first follower and the second follower slide over the rail as they are advanced or retracted along the first threaded portion and the second threaded portion.
Example 9 – The catheter articulation system of any of the preceding examples, further comprising a first bearing and a second bearing and configured to receive and support the lead screw.
Example 10 – The catheter articulation system of any of the preceding examples, wherein the first follower and the second follower are configured to engage tabs of a catheter to move the tabs towards or away from one another to articulate the catheter in a first plane.
Example 11 - A navigational catheter system, including a catheter drive system configured to receive a catheter the catheter drive system having a holder, a motor configured to be secured to the holder, a lead screw connected to the motor and including a first threaded portion having a first major diameter and a second threaded portion having a second smaller major diameter, a first follower having internal threads configured to advance or retract along the first threaded portion, a second follower having internal threads configured to advance or retract along the second threaded portion, a mount connected to the catheter drive system and configured to enable adjustment of an orientation of the catheter drive system, and a base, removably connectable to the mount, the base enabling adjustment of a position of the catheter drive system.
Example 12 - The navigational catheter system of example 11, wherein the first follower is advanceable over the second threaded portion without interacting with the threads of the second threaded portion.
Example 13 – The navigational catheter system of any of examples 11-12, wherein rotation of the lead screw advances the first follower along the first threaded portion and the second follower along the second threaded portion a same longitudinal distance.
Example 14 – The navigational catheter system of any of examples 11-13, wherein rotation of the lead screw in a first direction advances the first follower along the first threaded portion in a first direction and advances the second follower along the second threaded in a second direction opposite the first direction to articulate the catheter in first direction in a first plane.
Example 15 – The navigational catheter system of any of examples 11-14, wherein rotation of the lead screw in a second direction advances the first follower along the first threaded portion in a second direction and advances the second follower along the second threaded in a first direction opposite the second direction to articulate the catheter in a second direction in the first plane.
Example 16 – The navigational catheter system of any of examples 14-15, wherein the first follower and the second follower are configured to engage tabs of the catheter to move the tabs towards or away from one another to articulate the catheter in the first direction or the second direction in the first plane.
Example 17 – The navigational catheter system of any of examples 11-16, wherein the second threaded portion has a different number of starts than the first threaded portion.
Example 18 - The navigational catheter system of any of examples 11-17, wherein the second threaded portion has a different pitch than the first threaded portion.
Example 19 - The navigational catheter system of any of examples 11-18, further comprising a rail secured in the holder, wherein the first follower and the second follower slide over the rail as they are advanced or retracted along the first threaded portion and the second threaded portion.
Example 20 - The navigational catheter system of any of examples 11-19, further comprising a single bearing configured to receive and support the lead screw.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules without departing from the scope of the disclosure.

We claim:

1. A catheter articulation system comprising:
a holder;
a motor configured to be secured to the holder;
a lead screw connected to the motor and including a first threaded portion having a first major diameter and a second threaded portion having a second smaller major diameter;
a first follower having internal threads configured to advance or retract along the first threaded portion; and
a second follower having internal threads configured to advance or retract along the second threaded portion.

2. The catheter articulation system of claim 1, wherein the first follower is advanceable over the second threaded portion without interacting with the threads of the second threaded portion.

3. The catheter articulation system of claim 1, wherein rotation of the lead screw advances the first follower along the first threaded portion and the second follower along the second threaded portion a same longitudinal distance.

4. The catheter articulation system of claim 1, wherein rotation of the lead screw in a first direction advances the first follower along the first threaded portion in a first direction and advances the second follower along the second threaded in a second direction opposite the first direction.

5. The catheter articulation system of claim 1, wherein rotation of the lead screw in a second direction advances the first follower along the first threaded portion in a second direction and advances the second follower along the second threaded in a first direction opposite the second direction.

6. The catheter articulation system of claim 1, wherein the second threaded portion has a different number of starts than the first threaded portion.

7. The catheter articulation system of claim 1, wherein the second threaded portion has a different pitch than the first threaded portion.

8. The catheter articulation system of claim 1, further comprising a rail secured in the holder, wherein the first follower and the second follower slide over the rail as they are advanced or retracted along the first threaded portion and the second threaded portion.

9. The catheter articulation system of claim 1, further comprising a first bearing and a second bearing and configured to receive and support the lead screw.

10. The catheter articulation system of claim 1, wherein the first follower and the second follower are configured to engage tabs of a catheter to move the tabs towards or away from one another to articulate the catheter in a first plane.

11. A navigational catheter system, comprising:
a catheter drive system configured to receive a catheter, the catheter drive system including:
a holder
a motor configured to be secured to the holder;
a lead screw connected to the motor and including a first threaded portion having a first major diameter and a second threaded portion having a second smaller major diameter;
a first follower having internal threads configured to advance or retract along the first threaded portion;
a second follower having internal threads configured to advance or retract along the second threaded portion;
a mount connected to the catheter drive system and configured to enable adjustment of an orientation of the catheter drive system; and
a base, removably connectable to the mount, the base enabling adjustment of a position of the catheter drive system.

12. The navigational catheter system of claim 11, wherein the first follower is advanceable over the second threaded portion without interacting with the threads of the second threaded portion.

13. The navigational catheter system of claim 11, wherein rotation of the lead screw advances the first follower along the first threaded portion and the second follower along the second threaded portion a same longitudinal distance.

14. The navigational catheter system of claim 11, wherein rotation of the lead screw in a first direction advances the first follower along the first threaded portion in a first direction and advances the second follower along the second threaded in a second direction opposite the first direction to articulate the catheter in first direction in a first plane.

15. The navigational catheter system of claim 11, wherein rotation of the lead screw in a second direction advances the first follower along the first threaded portion in a second direction and advances the second follower along the second threaded in a first direction opposite the second direction to articulate the catheter in a second direction in the first plane.

16. The navigational catheter system of claim 15, wherein the first follower and the second follower are configured to engage tabs of the catheter to move the tabs towards or away from one another to articulate the catheter in the first direction or the second direction in the first plane.

17. The navigational catheter system of claim 11, wherein the second threaded portion has a different number of starts than the first threaded portion.

18. The navigational catheter system of claim 11, wherein the second threaded portion has a different pitch than the first threaded portion.

19. The navigational catheter system of claim 11, further comprising a rail secured in the holder, wherein the first follower and the second follower slide over the rail as they are advanced or retracted along the first threaded portion and the second threaded portion.

20. The navigational catheter system of claim 11, further comprising a single bearing configured to receive and support the lead screw.
